(19) United States
(12) Patent Application Publication
(10) Pub. No.: US 2013/0018016 A1
(43) Pub. Date: Jan. 17, 2013

(54) Agent for Combating Ectoparasites

(71) Applicant: Henning Ueck, Bekmunde (DE)

(72) Inventor: Henning Ueck, Bekmunde (DE)

(73) Assignee: G. POHL-BOSKAMP GMBH & CO. KG, Hohenlockstedt (DE)

(21) Appl. No.: 13/400,391

(22) Filed: Feb. 20, 2012

Related U.S. Application Data
(63) Continuation of application No. 12/841,514, filed on Jul. 22, 2010, now abandoned.

(30) Foreign Application Priority Data
Jul. 24, 2009 (DE) 10 2009 034 704.6

(51) Int. Cl. A61K 31/695; A61P 33/14

(52) U.S. Cl. 514/63

(57) ABSTRACT

Disclosed is a composition for killing ectoparasites and/or their eggs, comprising at least one volatile, at room temperature liquid, non-polar organic solvent, 1 to 10 wt.-%, based on the total composition, of at least one spreading agent; and 35 to 65 wt.-%, based on the total composition, of at least one polysiloxane having a viscosity of greater than 90 cSt.

### CROSS-REFERENCE TO PRIOR APPLICATIONS

This application claims priority to and the benefit of German Patent Application No. 10 2009 034 704.6, filed Jul. 24, 2009, the contents of which are incorporated by reference herein.

### FIELD OF THE INVENTION

The present invention pertains to compositions for combating ectoparasites, in particular head lice and body lice, as well as their eggs.

### BACKGROUND OF THE INVENTION

Ectoparasites, such as lice, are parasites of the skin which are preferably located in very hairy regions. There, the female species lay ova which are encased in protective sheaths adhered to the hairs; in the case of lice these ova are also referred to as nits. Eggs of ectoparasites are often encased in protective sheaths which cannot be attacked by many toxic agents.

Neither lice nor nits can be removed by a normal hair wash, only a local treatment with suited agents, which mostly contain insecticides, can be successful to remove lice and/or nits. These active ingredients are very efficient, however, they are of toxicological concern and recently the development of resistances against a part of these agents has been observed.

Various cosmetics and medicinal products for combating ectoparasites have been developed in the past as an alternative to drug products, however, many of these products proved to be less or non-effective. A promising approach is the use of siloxane polymers for combating ectoparasites. For instance, the German patent application DE 28 235 95 discloses compositions for controlling lice and/or their eggs, comprising an amount of linear siloxane polymers, in particular dimethicones. It is explicitly stated in said patent application that only dimethicones having a viscosity in the range from 100 to 1000 cSt are most efficient in combating lice.

European patent EP 1 215 965-B1 discloses compositions for combating arthropods comprising two different silicones, one of which is a volatile siloxane polymer and the other is a non-volatile siloxane polymer. The volatile siloxane is preferably a cyclic siloxane such as cyclopentasiloxane or cyclomethicone, which is used in amounts of 95.5-97.5 wt.-%, based on the composition.

International patent application WO 2007/104345 discloses a composition for combating ectoparasites and/or their eggs comprising a low-viscosity polysiloxane, a higher-viscosity polysiloxane, and at least one spreading agent. The spreading agent provides for even distribution of the active ingredient and good penetration of the hair. The viscosity of the low-viscosity siloxane must be less than 10 cSt, and the viscosity of the higher-viscosity siloxane must be greater than 90 cSt. In a particular embodiment a formulation is disclosed, which comprises 30 to 49 wt.-% dimethicone having a viscosity of 1 cSt, 35 to 65 wt.-% dimethicone having a viscosity of 100 cSt, and 2 to 10 wt.-% medium-chain triglycerides and/or 1 to 5 wt.-% jojoba wax, each amount based on the total composition. It is assumed that the low-viscosity siloxane is absolutely necessary for the removal of the ectoparasites.

While the prior art compositions are satisfactory in combating lice, not all of them have a sufficient ovicidal efficacy, i.e. they do not destroy nits reliably. Another problem with the above-cited compositions is that volatile cyclic and linear polysiloxanes are highly flammable, and they must not be used near ignition sources. Furthermore, some volatile polysiloxanes are potentially hazardous to health.

Consequently, there is still a need for toxicologically acceptable but highly effective compositions for combating ectoparasites and their eggs that are safe and easy to apply.

### DETAILED DESCRIPTION OF THE INVENTION

In the studies underlying the present invention it was surprisingly found that compositions that contain a higher-viscosity linear polysiloxane, a spreading agent, and a readily volatile organic solvent that is a liquid at room temperature, which is preferably a hydrocarbon, exhibit just as high efficacy against both lice and nits as compositions comprising besides the higher viscosity siloxane and the spreading agent a larger amount of a low-viscosity siloxane.

The compositions according to the invention have very low surface tension and, at the same time, a very high spreadability, an excellent compatibility to skin and no risk to health. Without wishing to be bound to any theory, it is assumed that the compositions, due to their very good creep properties, are able to penetrate deeply into the tracheas and tracheoles of adult head lice and their larvae, or into the aerodynes of nits. There, the readily volatile hydrocarbon solvent used according to the invention evaporates, whereupon the composition in the tracheas and the nits, respectively, becomes more and more thick and eventually clots. As a consequence, the lice, larvae and nits are separated from gas exchange and suffocate.

The compositions according to the invention comprise a linear siloxane having a viscosity of greater than 90 cSt, at least one spreading agent, and a volatile, at room temperature liquid, non-polar organic solvent, that preferably is a C8-C18 hydrocarbon. According to a preferred embodiment, the organic solvent is a non-polar C8-C18 hydrocarbon selected from the group consisting of dodecane, isododecane, isotetradecane, isohexadecane, 2,5-dimethyl decant, and mixtures thereof, preferably selected from the group consisting of isodecane, isohexadecane, and mixtures thereof. Most preferred is the embodiment in which the organic solvent is isododecane.

According to a preferred embodiment the composition contains linear dimethicone having a viscosity of 100 cSt, medium-chain triglycerides, jojoba oil, and isododecane. The polysiloxane is contained in an amount of 35 to 65 wt.-%, preferably in an amount of 40, 45, 50, 55 or 55 wt.-%, the spreading agent is contained in an amount of 1 to 10 wt.-%, preferably in an amount of 2, 3, 4, 5, 6, 7, 8, or 9 wt.%, and the volatile, non-polar, at room temperature liquid, organic solvent is contained in an amount of 30 to 49 wt.-%, particularly preferred in an amount of 35, 40, 42 or 45 wt.%. The compositions according to the invention preferably contain from 2 to 10 wt.-% medium-chain triglycerides and/or 1 to 5 wt.-% jojoba wax. Each data given in wt.-% are based on the whole weight of the composition.

As spreading agent the compositions according to the invention preferably contain medium-chain triglycerides, coconut oil, palm kernel oil, babassu oil, jojoba oil, jojoba wax, cetearyl isononanoate, cetearyl octanoate, isopropyl myristate, isopropyl palmitate, ethylhexyl palmitate, and cocoyl caprylocaprate.

Additionally, the compositions may contain common cosmetic ingredients such as fragrances or common skin-care components. The following examples illustrate the invention in more detail.

The viscosity data of the present invention refer to a temperature of 25° C.

### EXAMPLES

Compositions containing the ingredients listed in the Table 1 were produced.

TABLE 1

Percentage by Weight

| Ingredients | Test | Comp. |
|---|---|---|
| Medium-chain triglycerides | 5.00 | 5.00 |
| Jojoba oil | 2.00 | 2.00 |
| α-Terpineol | 1.00 | 1.00 |
| Corymbia citriodora | 0.05 | 0.05 |
| Dimethicone, 100 cSt | 50.00 | 50.00 |
| Dimethicone, 1 cSt | — | 41.95 |
| Isododecane | 41.95 | — |
| Total Individual Substances | 100.00 | 100.00 |

The compositions according to the invention (Test) and the comparative compositions (Comp.) were examined with respect to their efficacy in killing lice and their ova. For this purpose the so-called dip method was used, in which adult body lice were completely immersed into the respective test solution. After taking the lice out off the test solution and an additional impact time of five minutes, the test solution was removed from the lice by rinsing with tap water for one minute. In a control procedure the lice were treated with tap water instead of test solution. Fifty lice were used for each test solution.

After the end of the tests described above, the vitality of the body lice was studied under a stereomicroscope at defined points of time (10 min, 20 min, 30 min, 1 h, 2 h, 4 h, 6 h, 12 h, 18 h, 24 h, 42 h and 48 h after treatment). At each observation time, the vitality of the lice was assessed based on pre-established, strictly defined criteria as shown in Table 2:

TABLE 2

| | |
|---|---|
| Fully active or | Vital and fully mobile lice with normal movements. |
| vital: | |
| Clearly vital: | Lice cannot move along in a coordinated manner or |
| | cannot get up from their supine position. |
| Slightly vital | Lice are incapable of running. Gut motility, feeler |
| (“no major | or leg movements (with or without stimulation with |
| vital signs”): | tweezers) still occur. |
| No vital signs | Lice display no vital signs even after stimulation |
| (=motionless): | with tweezers (no visible movements and no gut |
| | motility within one minute of observation). |

During the observation period, the lice were held without additional feeding in a plastic petri dish covered with wet filter paper.

The results of these experiments are summarized in Table 3 showing the percentages of lice that have died at the first observation point, i.e., 10 minutes after the solutions were rinsed off.

TABLE 3

| Formula | Dead Lice [%] |
|---|---|
| Test | 100 |
| Comparison | 100 |
| Control | 0 |

It was shown that the composition according to the invention has very good lice- and egg-killing efficacy that is comparable to that of the comparative composition containing a low-viscosity polysiloxane instead of isododecane. Moreover, the composition according to the invention has a lower flammability and an even better skin compatibility than the comparison composition.

1. A composition for killing ectoparasites or their ova, comprising at least one volatile, at room temperature liquid, non-polar organic solvent, 1 to 10 wt.-%, based on the total composition, of at least one spreading agent and 35 to 65 wt.-%, based on the total composition, of at least one polysiloxane having a viscosity of greater than 90 cSt.

2. The composition of claim 1, wherein the volatile, at room temperature liquid, non-polar organic solvent is contained in an amount of 30 to 49 wt.-%, based on the total composition.

3. The composition of claim 1, wherein the volatile, at room temperature liquid, non-polar organic solvent is a linear or branched hydrocarbon that comprises 8 to 18 carbon atoms.

4. The composition of claim 1, wherein the volatile, at room temperature liquid, non-polar organic solvent is selected from a group consisting of dodecane, isododecane, isotetradecane, isohexadecane, 2,5-dimethyl decane, and mixtures thereof.

5. The composition of claim 1, wherein the volatile organic solvent is isododecane.

6. The composition of claim 1, wherein the spreading agent is selected from medium-chain triglycerides, coconut oil, palm kernel oil, babassu oil, jojoba oil, jojoba wax, cetearyl isononanoate, cetearyl octanoate, isopropyl myristate, isopropyl palmitate, ethylhexyl palmitate, and cocoyl caprylocaprate.

7. The composition of claim 1, wherein 2 to 10 wt.-% medium-chain triglycerides and 1 to 5 wt.-% jojoba oil, each based on the total composition, are contained as a spreading agent.

8. The composition of claim 1, wherein the polysiloxane is dimethicone having a viscosity of 100 cSt.

9. A composition for killing ectoparasites, the composition comprising:
a polysiloxane having a viscosity greater than 90 cSt, 35 to 65 wt. %;
a spreading agent, 1 to 10 wt. %; and
a branched hydrocarbon, 30 to 49 wt. %,
based on the total composition.

10. The composition of claim 9, wherein the polysiloxane is dimethicone.

11. The composition of claim 10, wherein the dimethicone has a viscosity of 100 cSt.

12. The composition of claim 9, wherein the spreading agent comprises medium chain triglycerides.

13. The composition of claim 12, wherein the composition comprises jojoba oil as a further spreading agent.

14. The method of claim 13, wherein the composition comprises 2 to 10 wt. % medium-chain triglycerides and 1 to 5 wt. % joboba oil, based on the total composition.

15. The composition of claim 9, wherein the branched hydrocarbon is a C₈-C₁₈ hydrocarbon.

16. The composition of claim 9, wherein the hydrocarbon is liquid at room temperature.

17. The composition of claim 9, wherein the branched hydrocarbon is isododecane.

18. The composition of claim 9, wherein the ectoparasite is lice.

19. The composition of claim 10, wherein the composition kills lice ova.

20. A composition for killing ectoparasites, the composition comprising:
medium chain triglycerides, 5 wt. %;
jojoba oil, 2 wt. %;
α-terpineol, 1 wt. %;
corymbia citriodora, 0.5 wt. %;
dimethicone, 100 cSt., 50 wt. %; and
isododecane, 41.95 wt. %,
based on the total composition.
